(19) United States
(12) Patent Application Publication
(10) Pub. No.: US 2025/0134552 A1
(43) Pub. Date: May 1, 2025

(54) APPARATUS AND METHOD TO FACILITATE INTRODUCTION OF CERVICAL BALLOON CATHETER

(71) Applicant: The Feinstein Institutes for Medical Research, Manhasset, NY (US)

(72) Inventors: Rachel Solmonovich, Manhasset, NY (US); Todd Goldstein, Albertson, NY (US); Allison Neuwirth, Long Beach, NY (US)

(21) Appl. No.: 18/927,416

(22) Filed: Oct. 25, 2024

Related U.S. Application Data
(60) Provisional application No. 63/546,456, filed on Oct. 30, 2023.

(51) Int. Cl. A61B 17/42; A61B 17/00

(52) U.S. Cl. CPC A61B 17/42; A61B 2017/00438; A61B 2017/4225

(57) ABSTRACT

A device facilitates placement of a balloon catheter to provide mechanical ripening of the cervix. The device is fitted over the gloved hand and index finger of a practitioner so that a shaft is held against the hand by a loop around the index finger and a tip portion holds the distal tip of the device to the practitioner's fingertip. The shaft and the tip portion are connected by a flexible portion that lies along the palmer side of the index finger. The shaft, flexible portion, and tip portion define an insertion path along the hand and finger. The flexible portion allows the practitioner to articulate the tip portion so that the os is located tactilely. The tip portion is advanced through the external os and a balloon catheter is advanced along the insertion path. The balloon portion of the catheter is advanced past the cervix and inflated to induce mechanical ripening of the cervix. The device is removed from the tube of the catheter by moving the catheter radially away from the insertion path through gaps in the shaft, finger loop and tip portion.

The present application claims priority under 35 U.S.C. § 119 to U.S. Provisional Patent Application No. 63/546,456, filed on Oct. 30, 2023, which is incorporated herein by reference.

### BACKGROUND

### Field

This disclosure relates to apparatus to facilitate the introduction of a balloon catheter through the cervix to promote cervical ripening and to induce labor. In particular, the disclosure relates to apparatus that fits over the hand and finger of a medical practitioner and facilitates insertion of a balloon catheter through the cervix.

Vaginal childbirth normally requires that the mother's body generate hormones that signal the cervix to soften, become thinner, and dilate. The pregnant woman's body generates endogenous hormones, including oxytocin, causing the cervix to ripen. Ripening of the cervix in turn releases hormones that cause the uterus to begin contractions. In some cases, the cervix does not respond sufficiently to these endogenous hormones, slowing the progress of labor.

Labor may be facilitated by the administration of drugs such exogenous oxytocin, which causes the uterus to begin contractions in the hopes of an eventual vaginal delivery. In general, the administration of drugs like oxytocin are more effective once the cervix has sufficiently ripened and partially dilated. In some pregnancies, such as those where a mother wishes to deliver vaginally after a previous cesarean birth (Vaginal Birth After Cesarian or VBAC), contractions induced by oxytocin or other drugs or hormones may result in intense contractions that may rupture the uterus that has been weakened by a previous cesarian procedure. In such situations, mechanical induction may be required. Even in non-VBAC pregnancies, before oxytocin can be administered, it is preferred that the cervix is at least partially dilated.

Labor may be facilitated by mechanical methods, for example, an amniotomy, where the amniotic sac is breached, or by ‘stripping the membranes” where a practitioner inserts a finger through the cervix and between the amniotic sac and the uterus. These methods cause the mother's body to excrete hormones that lead to labor. However, these techniques may not be sufficient to cause the cervix to fully ripen.

Balloon ripening is another mechanical technique for inducing and/or augmenting labor. A practitioner inserts a balloon catheter, such as a foley catheter, through the cervix so that the balloon portion of the catheter is beyond the inner os of the cervix. The balloon is then inflated. The balloon exerts force on the cervix, causing it to ripen and to dilate. Once the cervix is dilated greater than the diameter of the balloon, the balloon slides out through the cervix.

Balloon ripening may reduce the chance of tachysystole as compared with the administration of hormones such as Pitocin, oxytocin, or prostaglandin. In circumstances where hormones such as Pitocin or misoprostol are used to help ripen the cervix, combining that treatment with balloon ripening may make the hormone therapy more effective.

There are a number of problems with known apparatus and methods for balloon ripening. Where the cervix has only dilated a small amount (i.e., 0.5 centimeters (cm) to 1.0 cm) a practitioner may have difficulty inserting the catheter through the cervix. Blind placement of the catheter, that is, by holding the catheter while simultaneously locating the cervix tactilely, is technically challenging and often requires multiple attempts that are uncomfortable for the patient. During the blind placement of a catheter, the cervix is not dilated enough for the practitioner to hold the cervix in place with a finger and guide the catheter through the cervix at the same time. Therefore, the practitioner must remove his/her finger from the cervix so there is room for the catheter to pass through. This causes the practitioner to lose the necessary positioning of the cervix and to rely on blind placement of the catheter.

The cervix can also be directly visualized, allowing the practitioner to accurately position the catheter for insertion. Visualizing the cervix generally requires placing the pregnant woman in stirrups and using a speculum to open the vagina to allow a line of sight to the cervix. The cervix may then need to be grasped with ring forceps so that the catheter can be introduced. This procedure generally causes discomfort for the mother and may be time-consuming for the practitioner.

Balloon catheters and stylets used for the insertion of the balloon catheters are generally formed from flexible materials. One known device for balloon ripening is the Cook® Cervical Ripening Balloon (Cook Medical LLC). This device includes a pocket near its distal end that is fitted over the distal end of a stylet. The practitioner then advances the stylet and catheter to the cervix and through the inner and outer os. However, balloon catheters and stylets are not sufficiently firm and can bend such that the insertion of the catheter is more challenging. Moreover, the use of special purpose catheters, such as balloon catheters and stylets, may increase the expense for the procedure and may make the procedure less widely available. A less expensive foley catheter faces the same difficulty when attempting a blind placement through a minimally dilated cervix.

Thus, there is a need for a balloon catheter introduction apparatus that allows the practitioner to maintain the positioning of the cervix while guiding a catheter through the cervix improving comfort experienced by the patient, reducing time of insertion, and reducing costs.

### SUMMARY

According to one aspect of the disclosure, a device facilitates mechanical ripening of the cervix using a balloon catheter. The device is shaped to fit over the gloved hand of a medical practitioner and defines an insertion path along the practitioner's hand and index finger to guide the placement of a balloon catheter through the cervix. The device includes a shaft with a central opening defining a passageway. A flexible portion is connected with a distal end of the shaft and a tip portion is connected with the distal end of the flexible portion. A finger loop on the shaft holds the distal end of the shaft fixed to the practitioner's hand. The tip portion encircles the practitioner's fingertip. The flexible portion allows the practitioner's fingertip to move freely to palpate the cervix. Once the external os is located, the practitioner advances a balloon catheter along the insertion path, through the cervix and into the near uterus. The balloon is inflated to provide mechanical pressure on the cervix to promote ripening. According to another aspect, the device allows a practitioner to hold the distal portion of the catheter against their palm and index finger while maintaining a grasp on the cervix. Once the cervix has been located, the device allows the practitioner to slide the catheter through the cervix easily, potentially improving comfort, reducing time, and reducing costs by avoiding special purpose catheters and stylets.

Once the balloon is in place, the device is moved away from the cervix and along the tube of the catheter. Gaps are provided in the components defining the insertion path that allow the tube of the catheter to move radially away from the device.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a perspective view of a device for facilitating the introduction of a balloon catheter to induce cervical ripening according to an embodiment of the disclosure;

FIG. 2 is a detailed view of the device of FIG. 1;

FIGS. 3A, 3B, and 3C show use of the device of FIG. 1 to insert a balloon ripening catheter;

FIGS. 4A, 4B, 4C, and 4D show removal of the device of FIG. 1 from the tube of a catheter;

FIGS. 5A and 5B show the device of FIG. 1 provided with an extension portion according to an embodiment of the disclosure;

FIG. 6 shows a device for facilitating the introduction of a balloon catheter to induce cervical ripening according to another embodiment of the disclosure;

FIG. 7 shows the device of FIG. 6 attached to the hand of a medical practitioner;

FIG. 8 shows removal of the device of FIG. 6 from the tube of a catheter;

FIG. 9 shows the device of FIG. 6 provided with an extension portion according to an embodiment of the disclosure;

FIG. 10 shows a device for facilitating the introduction of a balloon catheter to induce cervical ripening according to another embodiment of the disclosure;

FIG. 11 shows a device for facilitating the introduction of a balloon catheter to induce cervical ripening according to another embodiment of the disclosure; FIG. 12 is a perspective view of a device for facilitating the introduction of a balloon catheter to induce cervical ripening according to another embodiment of the disclosure;

FIG. 13 shows the device of FIG. 12 associated with a balloon ripening catheter; and

FIG. 14A, 14B, and 14C show use of the device of FIG. 12 to insert a balloon ripening catheter.

### DETAILED DESCRIPTION

As used herein, the term “distal” refers to the direction toward a bodily structure being treated using apparatus according to embodiments of the disclosure. The term “proximal” refers to the direction away from the bodily structure being treated and towards a practitioner manipulating apparatus.

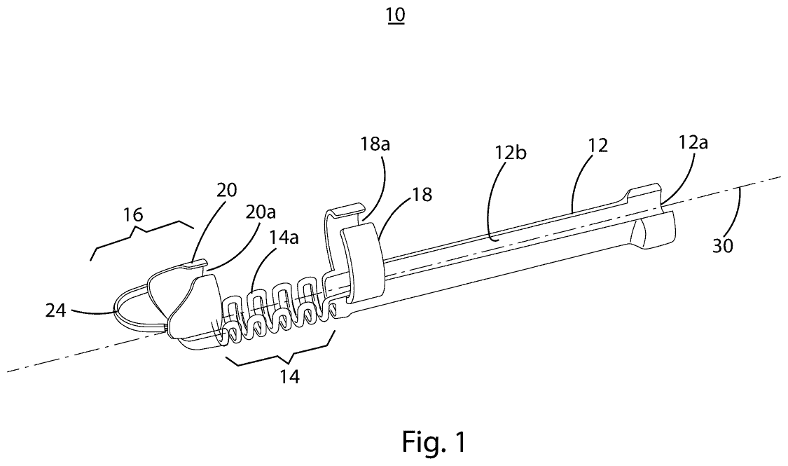

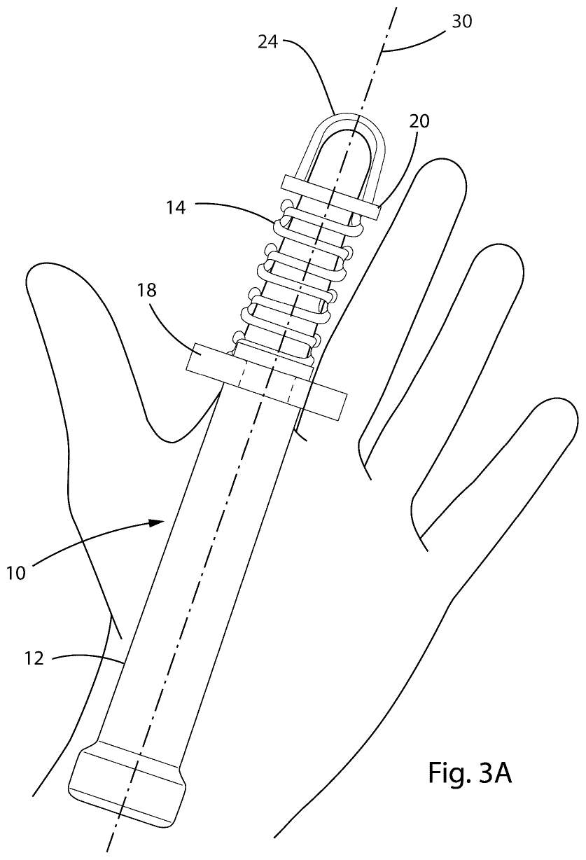

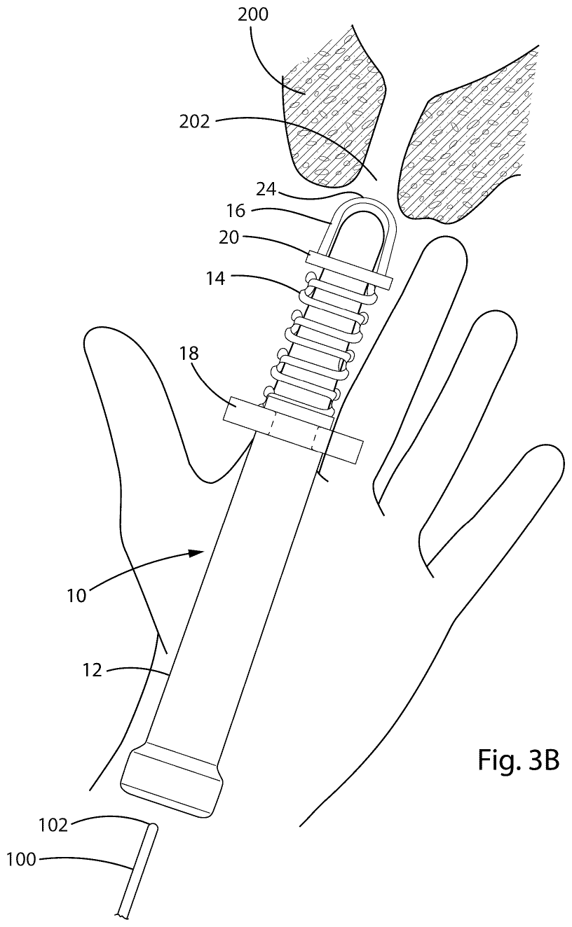

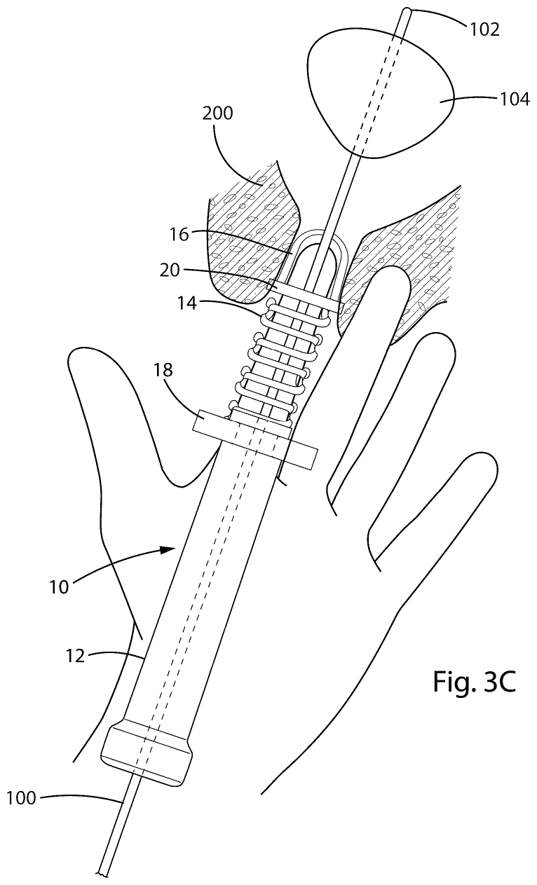

FIG. 1 shows a balloon placement device 10 according to an embodiment of disclosure. Device 10 is intended to assist a medical practitioner in placing a cervical ripening balloon catheter through the cervix to facilitate cervical ripening. Device 10 is sized and shaped to fit onto the gloved hand of the practitioner. FIG. 3A shows device 10 fitted to a hand and index finger.

As shown in FIG. 1, shaft 12 forms the proximal end of the device. Passageway 12a extends through the length of shaft 12. Passageway 12a is sized to permit a catheter, such as a foley catheter or other inflatable catheter, to move axially along shaft 12 without kinking. According to one embodiment, passageway 12a has a semicircular cross section with an opening 12b along one side. Opening 12b is sized to allow passage of the tube portion of the balloon catheter into and out from the interior of passageway 12a in a radial direction, as will be explained below.

Flexible portion 14 is connected with the distal end of shaft 12. Tip portion 16 is connected with the distal end of flexible portion 14. Device 10 defines an insertion path 30 through shaft 12, flexible portion 14, and tip portion 16. As will be explained below, a medical device, such as a foley catheter is advanced along insertion path 30.

Flexible portion 14 allows tip portion 16 to be freely articulated relative to shaft 12 by the index finger. According to one embodiment, flexible portion 14 is formed by a series of alternating loops 14a. The ends of the loops are separated by a gap. According to one embodiment, loops 14a define a semicircular cross section similar to the cross section of passageway 12a with a gap formed between loops 14a to allow the tube of a balloon catheter to pass radially into and out from the interior portion of flexible portion 14 along insertion path 30.

Fingertip cap 20 is provided on tip portion 16. Bow portion 24 is connected with the distal end of cap 20. As shown in FIG. 3A, when device 10 is positioned on a practitioner's hand, bow portion 24 extends across the tip of the finger. This arrangement holds tip portion 16 on the practitioner's fingertip and prevents the device from moving proximally as the practitioner's finger and the device are advanced through the cervix. Cap 20 includes a gap 20a sized to allow the tube of a catheter to be moved radially into and out from insertion path 30, as will be discussed below.

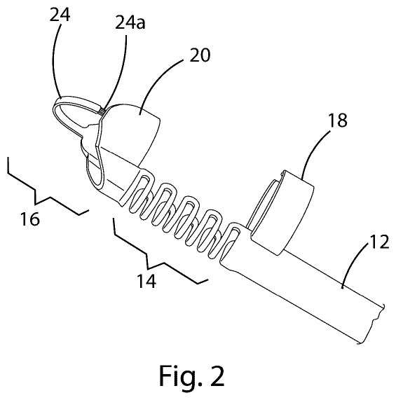

FIG. 2 is a detailed view of the distal portion of device 10. According to one embodiment, bow 24 is connected with cap 20 on either side of the fingertip. At least one such connection includes a break-away portion 24a. Breakaway portion 24a allows the practitioner to snap open bow 24 to allow device 10 to be removed from the tube of a catheter once the procedure is completed.

Finger loop 18 is provided near the distal end of passageway 12 proximal of flexible portion 14. According to this embodiment, finger loop 18 includes a gap 18a that is sized to allow the tube of a catheter 100 to be moved into and out from insertion path 30.

Finger loop 18 partially encircles the practitioner's finger proximal of the fingertip. According to one embodiment, finger loop 18 encircles the practitioner's finger about the proximal phalanx. Gap 18a is positioned on the dorsal side of the finger. Finger loop 18 provides a secure connection between shaft 12 and the practitioner's hand. Flexible portion 14 allows the practitioner to flex the index finger normally, with the tip portion 16 and distal end of shaft 12 fixed, respectively, to the tip and proximal portion of the finger.

Cap 20 partially encircles the practitioner's fingertip with gap 20a on the dorsal side of the fingertip. Bow 24 extends across the end of the practitioner's fingertip.

According to one embodiment, device 10 is formed by molding from medically suitable materials that are approved for use in the human body. According to another embodiment, device 10 is made from medically suitable material that is also suitable for sterilization and, optionally, repeated sterilization. A general example of suitable materials include a variety of hard and soft plastics used alone or in combination. More particularly, the following materials, alone or in any combination, can be used: including polyvinyl chloride, silicone, polyurethane, polyester, polyacrylate, polytetrafluoroethylene, polypropylene, polyethylene, polystyrene, polycarbonate, and the like. According to a preferred embodiment, device 10 is injection molded as a single piece.

FIGS. 3A to 3C show device 10 being used to deploy a cervical ripening balloon according to an embodiment of the disclosure. FIG. 3A shows device 10 fitted on the gloved hand of a medical practitioner. Shaft 12 lies along the palm. Insertion path 30 extends through passageway 12a. The palm of the hand covers opening 12b in shaft 12 to form a closed or partially close pathway through shaft 12. Flexible portion 14 lies along the palmer side of the practitioner's index finger with the opening of loops 14a against the practitioner's finger to encircle or partially encircle insertion path 30 through flexible portion 14. Flexible portion 14 allows freedom for the finger to bend, allowing the practitioner flexibility in locating cervix 200, which may be found anteriorly, posteriorly, or midline in the vagina. Once the cervix is palpated, as shown in FIG. 3C, the finger and device are inserted through the external and internal os. Bow portion 24 provides a curved surface that presses on the cervix to widen the opening.

In addition to facilitating the placement of a balloon catheter with a single balloon, such as a foley catheter, device 10, according to embodiments of the disclosure can facilitate the placement of catheters with multiple balloons. Where the catheter has two balloons, such as the Cook® Cervical Ripening Balloon, the device is introduced into the cervix, as described above. The two-balloon catheter is then advanced along the insertion path and through the cervix until the distal balloon is positioned in the near uterus and the proximal balloon is positioned outside of the external os. Both balloons are inflated to apply pressure to the cervix to promote ripening.

The practitioner fits tip 102 of catheter 100 into passage 12a and pushes the catheter along insertion 30 through shaft 12, flexible portion 14, through cap 20. The insertion path is partially defined by the gloved hand and index finger of the practitioner. This allows the practitioner to sense the location of the catheter relative to the cervix tactilely, without having to visualize the cervix.

According to some embodiments, insertion path 30 forms a close fit to the diameter of the catheter to limit the bend angle of the catheter as it is pushed through device 10. This may prevent catheter 100 from kinking. Tip 102 of catheter 100 advanced through the inner os to the near uterus. Balloon portion 104 of catheter 100 is then inflated, for example, by pumping fluid through the catheter, securing the balloon inside the uterus. The inflated balloon rests against the cervix, providing mechanical pressure to stimulate cervical ripening. The practitioner then pulls device 10 in the proximal direction along the tube of the catheter, removing the device from the cervix and vagina.

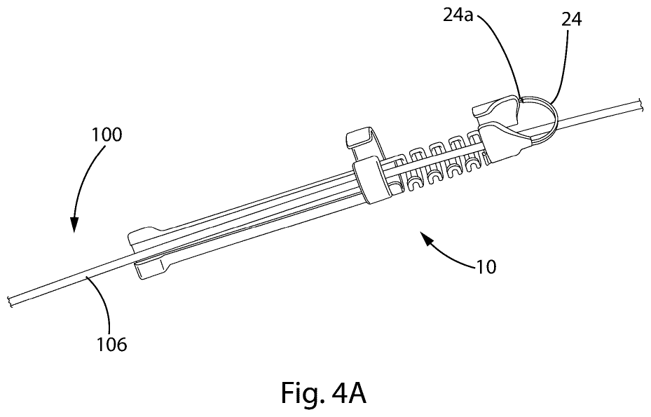

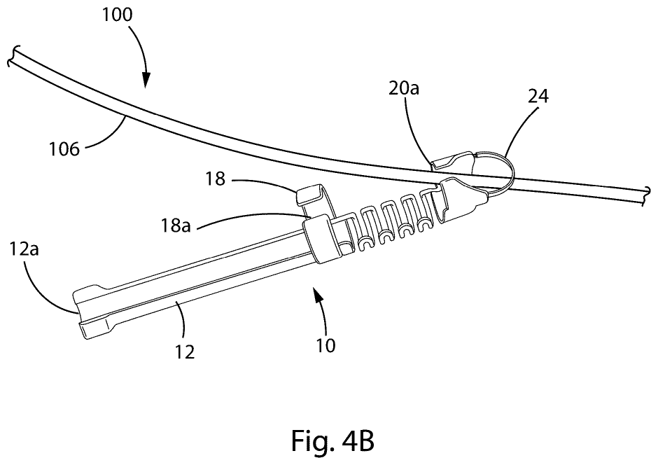

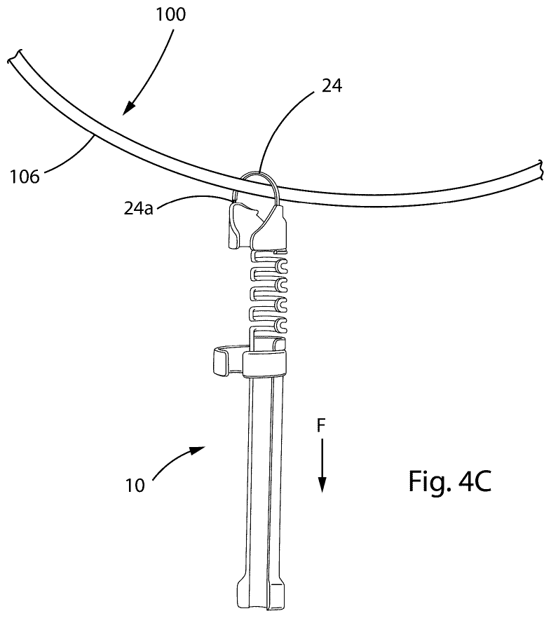

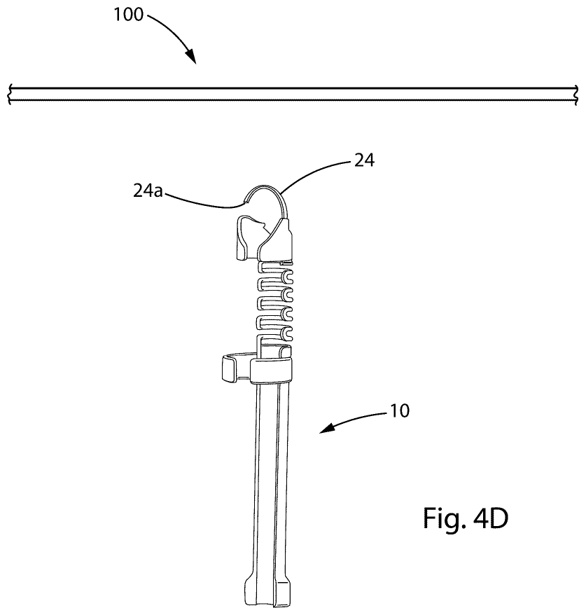

Device 10 is then removed from the tube of the catheter as shown in FIGS. 4A to 4D. As shown in FIG. 4A, device 10 has been withdrawn from the vagina. Tube 106 of Catheter 100 is positioned in passageway 12a, through flexible portion 14 and between cap 20 and bow portion 24. As shown in FIG. 4B, tube 106 is pulled radially outward from passageway 12a through opening 12b. Catheter 100 is removed from within finger loop 18 through gap 18a and removed from cap 20 through gap 20a.

As shown in FIG. 4C, device 10 is pulled away from tube 106. As shown in FIG. 4D, break-away portion 24a snaps, allowing device 10 to completely separate from catheter 100.

Device 10 may be particularly useful when the cervix is only dilated 0.5-1 cm, making it challenging to place the foley catheter blindly without a guide. Using device 10 may reduce or eliminate the discomfort to the patient caused by placing the patient's feet in stirrups, placing a speculum in the vagina, grasping the cervix with ringed forceps, and guiding the foley into the uterus under direct visualization.

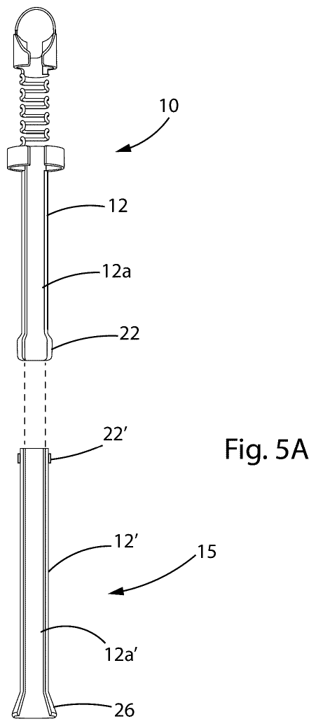

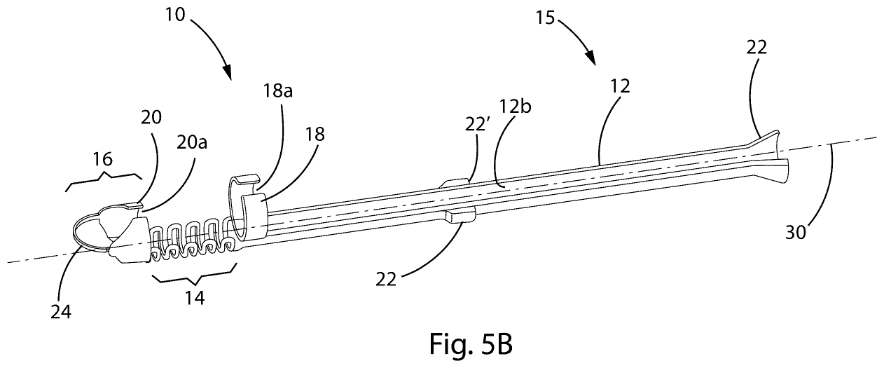

FIGS. 5A and 5B show extension portion 15 according to an embodiment of the disclosure that may be connected with device 10 to increase the distance between tip portion 16 in contact with the cervix and the proximal end. Depending on the pregnant woman's anatomy, such an extension may be needed so that the proximal opening 26 is easily visualized.

According to one embodiment, device 10 is the same as described above and shown in FIGS. 1 to 4D. According to another embodiment, device 10 includes device coupling 22. Passageway 12a and opening 12b pass through device coupling 22.

Extension portion 15 is formed by shaft 12′. Extension coupling 22′ is formed at the distal end of extension portion 10b. As shown in FIG. 5B, device coupling 22 and extension coupling 22′ connect with one another to join device 10 with extension portion 15. Shaft 12′ includes passageway 12a′. When extension portion 15 is connected with device 10, as shown in FIG. 5B, pathways 12a and 12a′ are aligned to increase the length of the insertion path. Proximal opening 26 at the proximal end of shaft 12′ is sized to accept insertion of catheter 100. According to one embodiment, proximal opening 26 is provided with a tapered flange to facilitate insertion of catheter tip 102 into and through extension portion 10b.

Only one extension portion 15 is described, but a greater number of extensions may be provided within the scope of the disclosure.

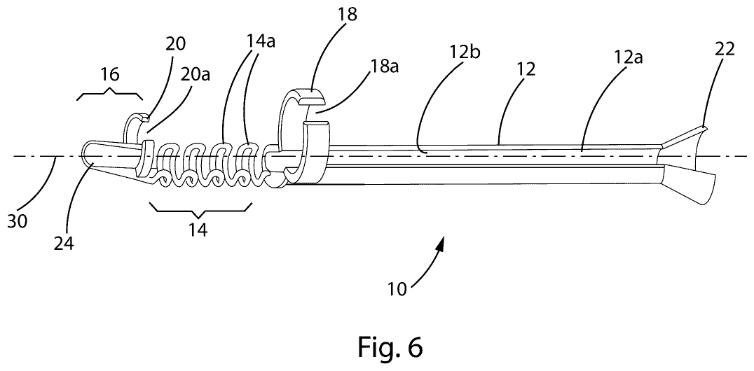

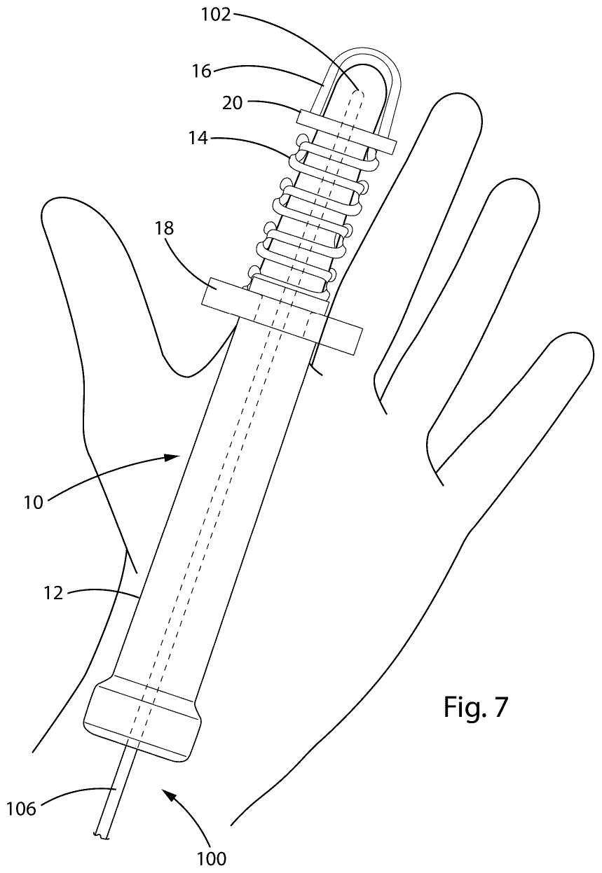

FIG. 6 shows a balloon placement device 10 according to another embodiment of disclosure. The same numbering is used to identify elements that are similar to previous embodiments. Device 10 is intended to assist a medical practitioner in placing a cervical ripening balloon catheter through the cervix to facilitate cervical ripening. Device 10 is sized and shaped to fit onto the gloved hand of the practitioner. FIG. 7 shows the device fitted to a gloved hand.

As shown in previous embodiments, shaft 12 forms the proximal end of the device. Passageway 12a extends through the length of shaft 12. Passageway 12a is sized to permit a catheter, such as a foley catheter or other inflatable catheter, to move axially along shaft 12 without kinking. According to one embodiment, passageway 12a has a semicircular cross section with an opening 12b along one side. Opening 12b is sized to allow passage of the tube portion of the balloon catheter into and out from the interior of passageway 12a, as will be explained below.

Flexible portion 14 is connected with the distal end of shaft 12. Tip portion 16 is connected with the distal end of flexible portion 14. Device 10 defines an insertion path 30 though shaft 12, flexible portion 14, and tip portion 16. As will be explained below, a medical device, such as a foley catheter is advanced along insertion path 30.

Flexible portion 14 allows fingertip portion 16 to be freely articulated relative to shaft 12. According to one embodiment, flexible portion 14 is formed by a series of alternating loops 14a separated by gaps. According to one embodiment, loops 14a define a semicircular cross section similar to the cross section of passageway 12a with a gap formed between loops 14a to allow the tube of a balloon catheter to pass into and out from the interior portion of flexible portion 14 along insertion path 30.

Tip portion includes a fingertip loop 20 at a proximal end of tip portion 16. Fingertip loop 20 includes fingertip gap 20a. Tapered portion 24 extends distally from fingertip loop 20.

Finger loop 18 is provided near the distal end of passageway 12 proximal of flexible portion 14. According to this embodiment, finger loop 18 includes a gap 18a sized to allow the tube of a catheter 100 to be moved into and out from insertion path 30.

Finger loop 18 partially encircles the practitioner's finger proximal of the fingertip. According to one embodiment, finger loop 18 encircles the practitioner's finger about the proximal phalanx. Gap 18a is positioned on the dorsal side of the finger. Finger loop 18 provides a secure connection between device 10 and the practitioner's hand. Flexible portion 14 allows the practitioner to flex the index finger normally, with the tip portion 16 and distal end of shaft 12 fixed, respectively, to the tip and proximal portion of the finger.

As shown in FIG. 7, tapered portion 24 rests against the practitioner's fingertip and defines a space for receiving tip 102 of catheter 100. Tapered portion 24 forms a ramped surface along the practitioner's fingertip. The tapered aspect of the practitioner's fingertip and tapered portion 24 help to open the os and to guide the distal part of device 10 through the cervix. This feature allows device 10 to effectively dilate the cervix enough to allow entry into the uterus. Once the finger and tip portion are fully through the cervix, catheter 100 is advanced along insertion path 30 to extend into the uterus. Device 10 is then withdrawn from the cervix and vagina and balloon portion 104 of catheter 100 is inflated, for example, by forcing saline solution through catheter 100 using a syringe. This secures the balloon in the uterus to provide mechanical stimulation to promote cervical ripening.

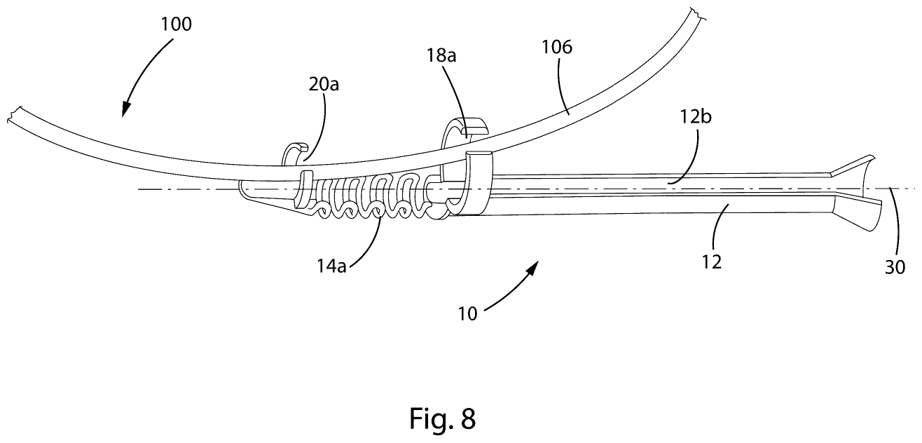

The practitioner then removes device 10 from their hand. Device 10 is then removed from tube 106 of catheter 100, as shown in FIG. 8. Catheter 100 exits passageway 12a through opening 12b. Catheter 100 likewise exits fingertip loop 20 and finger loop 18 through gaps 20a and 18a. This allows device 10 to be removed from catheter 100.

In the procedure described above, catheter tip 102 was positioned between tip portion 16 and the practitioner's fingertip prior to palpating the cervix, as shown in FIG. 7. According to another embodiment, device 10 is fitted to the practitioner's hand and index finger prior to engagement of catheter 100 along insertion path 30. The practitioner palpates the cervix and at least partially extends the fingertip and tip portion 16 through the cervix. Catheter tip 102 is then inserted through shaft 12 and advanced along insertion path 30 until the tip extends from the distal end of the device. A conical entrance portion 22 is provided at the proximal end of shaft 12 to facilitate insertion of the catheter.

The length of shaft 12 may be selected so that its proximal end extends outward of the vagina when tapered portion 16 is engaged with the cervix. This allows the practitioner to visualize the proximal end of shaft 12 to fit the catheter tip into shaft 12 and along insertion path 30.

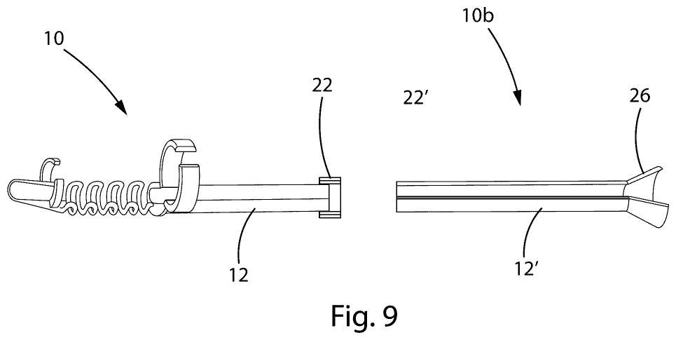

According to a further embodiment, shown in FIG. 9, shaft 12 includes device coupling 22 at the proximal end of shaft 12. Extension portion 10b is provided. Extension coupling 22′ is provided at the distal end of extension portion 10b. Couplings 22, 22′ connect extension portion 10b with device 10 to adjust the length of the insertion path through the device. When joined, device 10 and extension portion 10b define an insertion path similar to the insertion path discussed in previous embodiments. A proximal opening 26 is provided at the proximal end of extension portion 10b. According to one embodiment, proximal opening 26 includes a conical flange to facilitate insertion of tip 102 of catheter 100 into the device and along the insertion path.

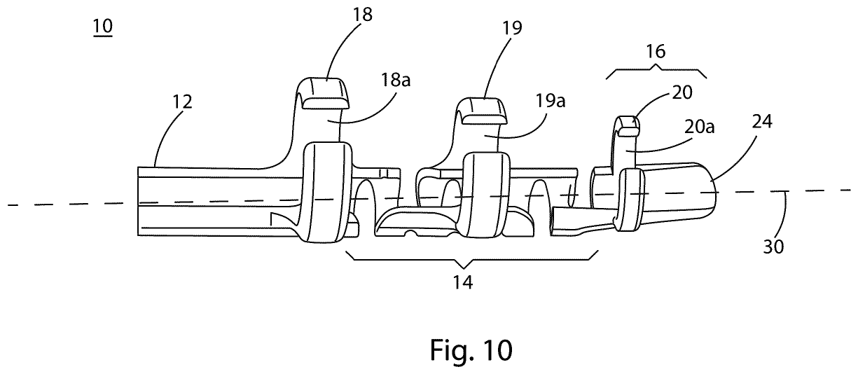

FIG. 10 shows device 10 according to another embodiment of the disclosure. The distal end of shaft 12 is connected with the proximal end of flexible portion 14 and tapered portion 16 is connected with the distal end of flexible portion 14, as in the previous embodiments. In this embodiment, three loops 18, 19, 20 that partially encircle the practitioner's finger are provided. Fingertip loop 20 and finger loop 18 are similar to the embodiments discussed above with respect to FIGS. 6 to 8. In addition, middle finger loop 19 is connected with flexible portion 14. According to this embodiment, device 10 is fitted over the gloved index finger, as discussed above, with loops 18, 19, 20 securing the device to the index finger. Fingertip loop 20 encircles the index finger at about the distal phalanx. Middle finger loop 19 encircles the finger at about the middle phalanx. Finger loop 18 encircles the finger at about the proximal phalanx. The portion of flexible portion 14 proximal of middle loop 19 extends across the practitioner's proximal interphalangeal joint and the portion of the flexible portion distal of the middle loop extends across the practitioner's distal interphalangeal joint.

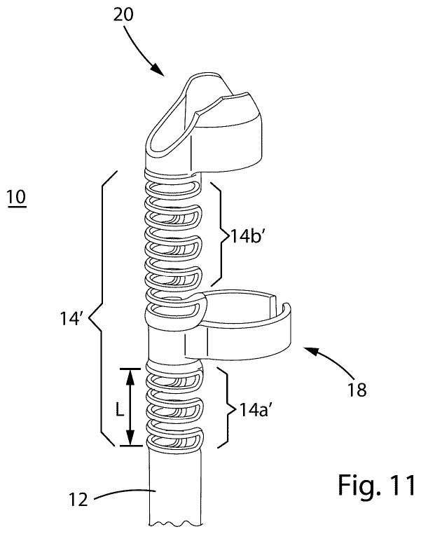

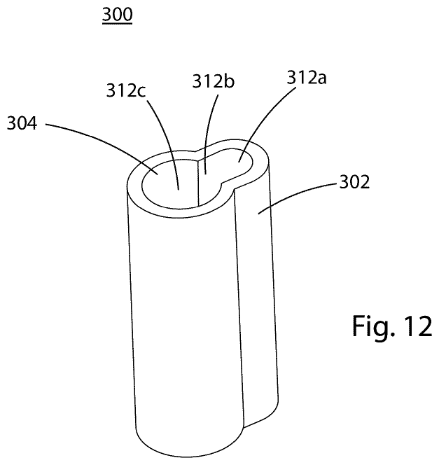

FIG. 11 shows a device 10 according to another embodiment of the disclosure with a configuration adapted to the practitioner's hand that may improve mobility while using the device to locate the cervical os. Flexible portion 14′ is formed by proximal and distal flexible portions 14a′, 14b′. Proximal flexible portion 14a′ connects proximal finger loop 18 to the shaft 12. Proximal portion 14a′ has a selected length designed to extend across the practitioner's metacarpophalangeal joint such that the practitioner experiences a greater degree of mobility of his/her hand while using the device. Distal flexible portion 14b′ couples proximal finger loop 18 with one or more additional finger loops. In the embodiment shown in FIG. 11, there are two finger loops 18, 20 that partially encircle the practitioner's finger. Fingertip loop 20 and finger loop 18 are similar to the embodiments discussed above with respect to FIGS. 6-8. Fingertip loop 20 encircles the index finger at about the distal phalanx. Finger loop 18 encircles the finger at about the proximal phalanx. However, a greater number of loops may be used, for example three finger loops as shown in FIG. 10 in which the third loop encircles the index finger about the intermediate phalanx.

Flexible member 14a′ connects the distal end of shaft 12 with finger loop 18. The flexible member 14a′ has a predetermined length “L” such that the flexible member 14a′ extends across the practitioner's metacarpophalangeal joint as the practitioner is wearing the device. Flexible portion 14b′ connects finger loop 18 with fingertip loop 20 such that flexible member 14b′ extends across the practitioner's distal interphalangeal joint.

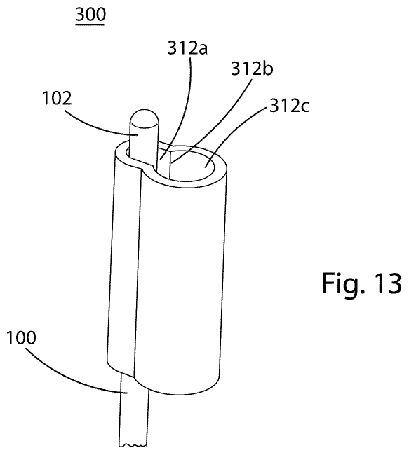

FIGS. 12-13 show device 300 according to another embodiment of this disclosure. Device 300 has an exterior surface 302 and an interior surface 304 that define two passageways 312a, 312c. In the exemplary embodiment shown, the two passageways 312a, 312c have a semicircular cross section with an opening 312b that connects the passageways 312a, 312c. Passageways 312a, 312c extend through the length of device 300.

Passageway 312a is sized to permit a catheter, such as a foley catheter or other inflatable catheter, to move axially through passageway 312a without kinking. Opening 312b is sized to allow passage of the tube portion of the balloon catheter into and out from the interior of passageway 312a in a radial direction.

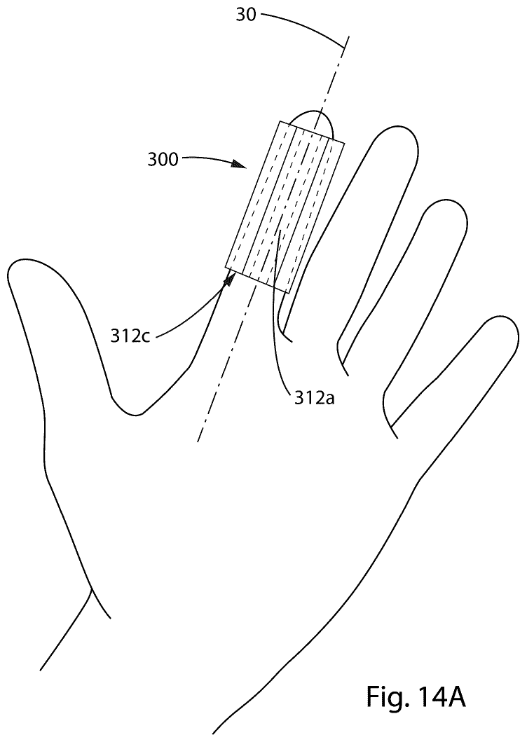

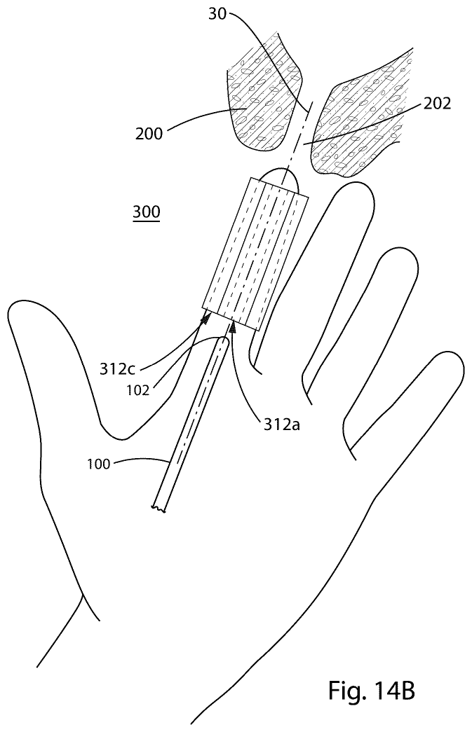

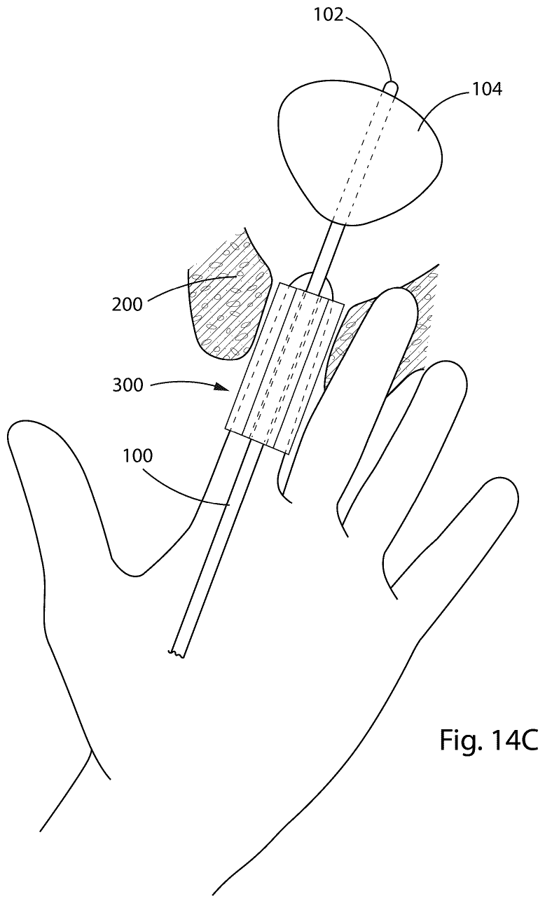

Passageway 312c is sized and shaped to fit onto the gloved hand of a practitioner. FIG. 14A shows device 300 fitted to an index finger of the practitioner such that a proximal and distal portion of the practitioner's finger does not reside within device 300. In another exemplary embodiment, device 300 is configured and dimensioned to extend generally the entire length of the practitioner's finger. In yet another embodiment, device 300 is configured and dimensioned such that only a portion of the distal phalange of the practitioner's finger resides within device 300. Device 300 is positioned so that passageway 312a is along the anterior surface of the practitioner's index finger.

According to one embodiment, device 300 is formed by molding from medically suitable materials that are approved for use in the human body. According to another embodiment, device 300 is made from medically suitable material that is also suitable for sterilization and, optionally, repeated sterilization. A general example of suitable materials include a variety of hard and soft plastics used alone or in combination. According to a preferred embodiment, device 300 is injection molded from silicone as a single piece. It is envisioned that device 300 may be sufficiently flexible, non-rigid, and/or pliable to permit the practitioner the ability to flex the index finger such that the practitioner may palpate the cervix.

FIGS. 14A to 14C show device 300 being used to deploy a cervical ripening balloon according to an embodiment of the disclosure. FIG. 14A shows device 300 fitted on the gloved hand of a medical practitioner. The practitioner's index finger is inserted through passageway 312c such that device 300 is secured substantially to the intermediate phalange with the practitioner's fingertip protruding from the device. However, device 300 may be secured substantially to the distal phalange or a combination of the distal, intermediate, and proximal phalanges. The anterior surface of the index finger covers opening 312b that connect passageways 312a and 312c to form a closed or partially closed pathway through passageway 312a. In an embodiment, device 300 has sufficient flexibility to allow freedom for the finger to bend, allowing the practitioner flexibility in locating cervix 200, which may be found anteriorly, posteriorly, or midline in the vagina. Once the cervix is palpated, as shown in FIG. 14C, the finger and device are inserted through the external and internal os. The natural curvature of the tip of the finger provides a curved surface that presses on the cervix to widen the opening.

In addition to facilitating the placement of a balloon catheter with a single balloon, such as a foley catheter, device 300, according to embodiments of the disclosure, can facilitate the placement of catheters with multiple balloons. Where the catheter has two balloons, such as the Cook® Cervical Ripening Balloon, the device is introduced into the cervix as described above. The two-balloon catheter is then advanced along the insertion path and through the cervix until the distal balloon is positioned in the near uterus and the proximal balloon is positioned outside of the external os. Both balloons are inflated to apply pressure to the cervix to promote ripening.

The practitioner fits tip 102 of catheter 100 into passageway 312a and pushes the catheter along insertion path 30 through passageway 312a. The insertion path is partially defined by the index finger of the practitioner. This allows the practitioner to sense the location of the catheter relative to the cervix tactilely, without having to visualize the cervix.

According to some embodiments, insertion path 30 forms a close fit to the diameter of the catheter to limit the bend angle of the catheter as it is pushed through device 300. This may prevent catheter 100 from kinking. Tip 102 of catheter 100 advanced through the inner os to the near uterus. Balloon portion 104 of catheter 100 is then inflated, for example, by pumping fluid through the catheter, securing the balloon inside the uterus. The inflated balloon rests against the cervix, providing mechanical pressure to stimulate cervical ripening. The practitioner then pulls device 300 in the proximal direction along the tube of the catheter, removing the device from the cervix and vagina. The practitioner then removes device 300 from their hand. Device 300 remains associated with the catheter external to the patient until the procedure is complete and the catheter is removed from the patient.

In the above description, the use of common reference numerals in different examples is for convenience only and is intended to refer to similar components and not necessarily identical components. While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

We claim:

1. A device for facilitating the insertion of a catheter through a cervix, comprising:
a shaft defining a passageway, wherein the passageway is sized to accept insertion of the catheter;
a finger loop connected with the shaft and positioned to connect the shaft with a finger of a practitioner;
a flexible portion connected with a distal end of the shaft; and
a tip portion connected with a distal end of the flexible portion,
wherein the tip portion is adapted to mechanically couple with a fingertip of the finger,
wherein the tip portion is shaped to be inserted at least partially through the cervix along with the fingertip,
wherein the flexible portion allows the tip portion to be articulated relative to the shaft by movement of the finger, and
wherein the passageway, the flexible portion, and the tip portion define an insertion path sized to accept passage of the catheter through the device.

2. The device of claim 1, wherein the shaft further comprises an opening along its length to allow the catheter to exit the passageway in a radial direction relative to the insertion path, wherein the finger loop comprises a first gap to allow the catheter to exit from within the finger loop in the radial direction, and wherein the tip portion includes a second gap to allow the catheter to exit the tip portion in the radial direction.

3. The device of claim 1, wherein the tip portion comprises a cap adapted to at least partially encircle the fingertip and a bow portion extending distal from the cap, wherein the bow portion is adapted to extend across fingertip.

4. The device of claim 3, wherein the bow portion includes a break-away segment.

5. The device of claim 1, further comprising an extension portion, wherein a distal end of the extension portion is adapted to connect with a proximal end of the shaft and wherein the insertion path extends through the extension portion.

6. The device of claim 2, wherein the flexible portion comprises one or more additional finger loops.

7. The device of claim 1, wherein the catheter comprises one or two balloons.

8. The device of claim 1, further comprising an intermediate loop connected with the flexible portion and positioned between the finger loop and the tip portion.

9. A device for facilitating the insertion of a catheter through a cervix, comprising:
a shaft defining a passageway;
a proximal flexible portion connected with a distal end of the shaft;
a proximal finger loop connected with a distal end of the proximal flexible portion;
a distal flexible portion connected with a distal side of the proximal finger loop; and
a distal finger loop connected with a distal end of the distal flexible portion,
wherein the passageway, proximal flexible portion, proximal finger loop, distal flexible portion, and distal finger loop define an insertion path sized to accept passage of the catheter through the device, and
wherein the proximal finger loop and distal finger loop are adapted to connect the device with the hand of a practitioner.

10. The device of claim 9, wherein the shaft further comprises an opening along its length to allow the catheter to exit the passageway in a radial direction relative to the insertion path, wherein the proximal and distal finger loops each comprise respective gaps to allow the catheter to exit from within the finger loops in the radial direction.

11. The device of claim 9, further comprising an extension portion, wherein a distal end of the extension portion is adapted to connect with a proximal end of the shaft and wherein the insertion path extends through the extension portion.

12. The device of claim 9, further comprising one or more additional flexible portions connecting one or more additional finger loops with a distal end of the distal finger loop.

13. The device of claim 9, wherein the catheter comprises one or two balloons.

14. A method of using the device of claim 2, the method comprising the steps of:
attaching the device to a finger of a medical practitioner, wherein the finger loop at least partially encircles the finger and wherein the tip portion at least partially encloses a tip of the finger to attach the tip portion to the fingertip;
articulating the tip portion by flexing the flexible portion to palpate the cervix to locate an external os of the cervix;
inserting the tip portion at least partially through the external os; and
advancing the catheter along the insertion path at least partially through the cervix.

15. The method of claim 8, wherein the finger loop is positioned to at least partially encircle the middle phalanx or the proximal phalanx of the finger.

16. The method of claim 8, wherein the catheter is a balloon catheter, further comprising the steps of:
further advancing the catheter to position a balloon portion of the catheter past an internal os of the cervix;
inflating the balloon portion;
moving the device in a distal direction along the catheter and away from the cervix; and
removing the catheter in the radial direction from the insertion path.

17. A device for facilitating the insertion of a catheter through a cervix, comprising:
an exterior surface and an interior surface defining a first and second passageway, wherein the first passageway is sized to accept insertion of a catheter and the second passageway is configured to accept the insertion of at least a portion of a finger of a practitioner.

18. The device of claim 12, wherein the first and second passageway have a semicircular cross section.

19. The device of claim 12, further comprising an opening that connects the first and second passageways, wherein the opening is sized such that a bend angle is limited to prevent kinking of the catheter.

20. The device of claim 12, wherein the device is sufficiently flexible to allow at least some flexion of a practitioner's finger.
